(19) United States
(12) Patent Application Publication
(10) Pub. No.: US 2024/0422470 A1
(43) Pub. Date: Dec. 19, 2024

(54) MODULAR EARPIECE

(71) Applicant: ODM GMBH, Wattenheim (DE)

(72) Inventors: Christopher KÜHN, Wattenheim (DE); David PFLÜGER, Wattenheim (DE)

(21) Appl. No.: 18/698,536

(22) PCT Filed: Sep. 28, 2022
(86) PCT No.: PCT/EP2022/077052

§ 371 Date: Apr. 4, 2024

(30) Foreign Application Priority Data
Oct. 4, 2021 (DE) 102021125704.2

(51) Int. Cl. H04R 1/10

(52) U.S. Cl. CPC H04R 1/1083; H04R 1/1008; H04R 1/1033; H04R 2201/107; H04R 2420/09; H04R 2460/01

(57) ABSTRACT

An earcup for headphones with a housing for receiving electroacoustic components and sound-absorbing material, wherein the housing has a housing base body for receiving electroacoustic components and a main shell module for receiving a sound-absorbing material, wherein the main shell module is mechanically connected to the housing base body, wherein a) the housing base body can be added to in a modular manner and a first housing module and a second housing module are provided, wherein the first housing module and the second housing module can each be connected mechanically and electrically to the housing base body alternatively as a different configuration stage and/or b) that the housing has the main shell module for providing different expansion stages, which is b1) is modularly interchangeable and at least one first shell replacement module is provided, which can be mechanically connected to the housing base body as an alternative to the main shell module and/or; b2) is modularly supplementable and at least one first shell replacement module is provided, wherein the first shell replacement module can be mechanically connected to the main shell module.

### FIELD OF THE INVENTION

The invention relates to an earcup for headphones with a housing for accommodating electroacoustic components and sound-absorbing material.

### BACKGROUND OF THE INVENTION

An earcup for headphones is already known from DE 1 209 163 B.

### SUMMARY OF THE INVENTION

The invention is based on the task of designing and arranging an earcup for headphones in such a way that simple manufacture and simultaneous cost savings are guaranteed.

The problem is solved in accordance with the invention in that the housing has a housing base body for accommodating electroacoustic components and a main shell module for accommodating a sound-absorbing material, wherein
a) the housing base body can be added to in a modular fashion and a first housing module and a second housing module are provided, wherein the first housing module and the second housing module can each be connected to the housing base body mechanically and electrically as an alternative expansion stage
b) that for providing different expansion stages the housing has the main shell module, which
b1) is modularly replaceable and at least one first shell replacement module is provided, which can be mechanically connected to the housing base body as an alternative to the main shell module;
b2) can be added in a modular manner and has at least one first shell replacement module, wherein the first shell replacement module can be mechanically connected to the main shell module.

This means that the use of housing modules or main shell modules and shell replacement modules enables a simple and favourable extension of the electronic and acoustic functionality of the earcup. The electronic functionality can include features such as Active Noise Cancelling (ANC) and/or Active Noise Reduction (ANR). Together with the shell replacement module, the main shell module ensures a change in the internal volume of the main shell module so that different sound attenuation properties of the earcup are guaranteed. It is therefore possible to switch between different expansion stages by changing or using the different modules.

The problem is also solved by a headphone consisting of an earcup and a second earcup, wherein the housing base body and/or the main shell module and/or the shell replacement modules of the two earcups have the same shape, in particular the same bearing and connection architecture.

It can also be advantageous if the housing base body and/or the main shell module and/or the shell replacement modules have a symmetry plane E and are mirror-symmetrical with respect to the symmetry plane E. The bearing and connection architecture, such as the mirror-symmetrical mounts for the speaker microphone or the mirror-symmetrical mounts for the headband, are of particular interest. The symmetry of the architecture or shape to be used ensures that the same housing base body or the same main shell module or shell replacement module can be used for the right or left earcup. The speaker microphone can also be positioned on both the right and left earcups. Design elements such as colour, colour pattern or lettering are excluded from the connection architecture.

For this purpose, it can be advantageous if the housing base body has a pair of bearing mounts for a headband and/or a pair of mounts for a speaker microphone to be arranged outside the housing base body, whereby the bearing mounts and/or the mounts are symmetrical to the symmetry plane E. This particular symmetry ensures a change of the earcup between a left and a right position on the ear and, independently of this, a change of the speaker microphone between a left and a right position. The speaker microphone is assigned a microphone input contact on or in the housing base body for connecting the speaker microphone.

It can also be advantageous if the housing modules have electro-acoustic components and the housing base body has electro-acoustic components, whereby the electro-acoustic components can be brought into active contact with the electro-acoustic components. This makes it possible to switch between different electroacoustic expansion stages. Various electro-acoustic or electronic functionalities are used, such as Active Noise Cancelling (ANC) and/or Active Noise Reduction (ANR).

Advantageously, a plug-in contact can be provided via which the active contact can be established. The plug-in contact therefore serves as an interface between the electroacoustic components of the housing module and the electroacoustic components of the housing base body.

It can be of particular importance for the present invention if the first housing module has at least one microphone as an electroacoustic component. Preferably, at least one pair of microphones is provided for ambient noise in order to reproduce a spatial impression of the noise environment.

In connection with the design and arrangement according to the invention, it can be advantageous if at least one second shell exchange module is provided which
a) can be mechanically connected to the housing base body as an alternative to the main shell exchange module or as an alternative to the first shell exchange module, or
b) can be mechanically connected to the first shell exchange module. This enables a wide variation of sound attenuation configuration levels.

It can also be advantageous if the main shell module has an overall height S0 and the first shell replacement module has an overall height S1, whereby the overall heights S0 and S1 are different. This allows a large variation in the overall heights of the sound attenuation or acoustic attenuation expansion stages.

It can also be advantageous if a second shell replacement module with a overall height S2 is provided, whereby the overall heights S0, S1 and S2 are different. By using the different heights, a wide range of expansion stages for acoustic damping can be achieved. The main shell module can therefore either be replaced by a shell replacement module of a different height or supplemented by a shell replacement module of the same height.

It can also be advantageous if the following applies for the overall heights S0, S1 and/or S2: S1=f1*S0 and/or S2=f2*S0, with X1<f1<Y1 and X2<f2<Y2. The range of expansion stages for acoustic damping can thus be optimally expanded. The respective main shell module, first shell replacement module or second shell replacement module contains sound attenuation material, whereby the sound attenuation material is provided in a thickness that corresponds to the overall heights S0, S1 and/or S2 of the corresponding module.

It can be advantageous if the housing base body has a plug socket for a plug of an electrical connection cable to a second earcup. The plug can be manually connected to or disconnected from the plug socket. Both earcups can be electrically connected via the connecting cable.

### BRIEF DESCRIPTION OF THE DRAWINGS

Further advantages and details of the invention are explained in the patent claims and in the description and shown in the figures. It shows:

FIG. 1 a perspective view of an earcup;

FIG. 2 top view of the earcup shown in FIG. 1;

FIG. 3a, 3b a perspective view of a shell exchange module;

FIG. 4 a schematic sketch of a headphone.

### DETAILED DESCRIPTION OF THE INVENTION

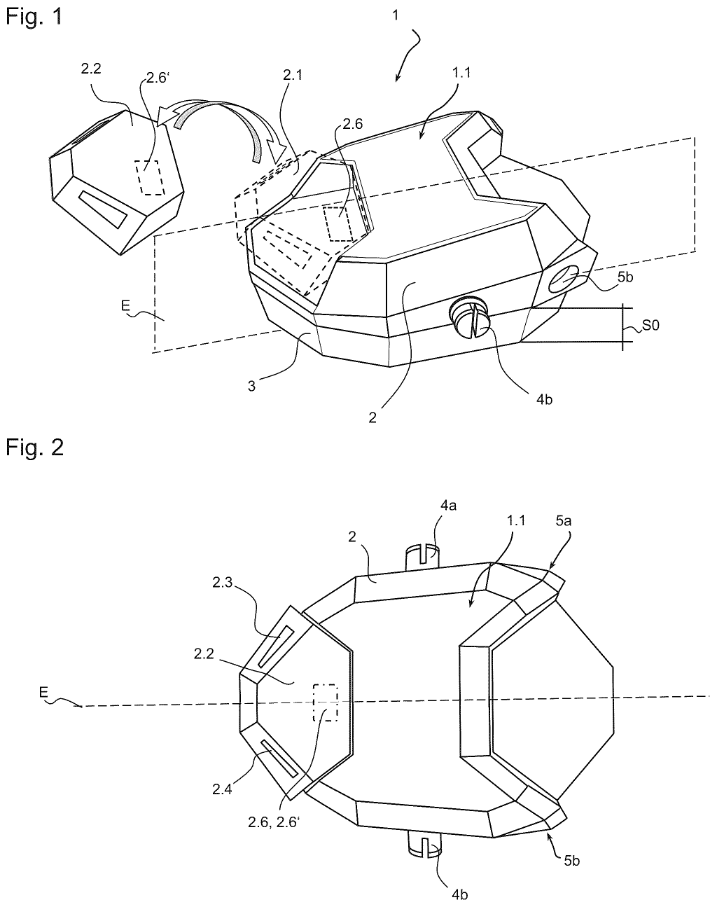

The earcup 1 shown in FIGS. 1 and 2 has a housing base body 2 and a main shell module 3. Together with the main shell module 3, the housing base body 2 forms the earcup 1. The earcup 1, i.e. the housing base body 2 and the main shell module 3, have a common symmetry plane E, to which the earcup 1 is mirror-symmetrical. The latter applies in particular to a bearing mount 4a, 4b for attaching the earcup 1 to a headband 9 and for the mounts 5a, 5b for a speaker microphone 6, as shown in FIG. 4.

Electroacoustic components such as loudspeakers are contained within the housing base body 2. The housing base body 2 is supplemented by a first housing module 2.1, which has additional electro-acoustic components such as microphones 2.3, 2.4 for ambient noise. The first housing module 2.1 is operatively connected to the housing base body 2 via a plug-in contact 2.6, 2.6′. Both the first housing module 2.1 and the housing base body 2 have a corresponding or interacting plug-in contact 2.6, 2.6′. The housing base body 2, together with the first housing module 2.1, form a first expansion stage of the earcup 1 with regard to the electroacoustic properties and possibilities of the earcup 1. As an alternative to the first housing module 2.1, a second housing module 2.2 can be used in a modular design. In this case, the first housing module 2.1 is replaced by the second housing module 2.2. The second housing module 2.2 can have further electro-acoustic properties, such as a noise cancellation or noise reduction function. The second housing module 2.2 has the same design as the first housing module 2.1 and is also electronically coupled to the housing base body 2 or its plug-in contact 2.6 via the plug-in contact 2.6′. A corresponding contact 2.6 is provided on the side of the respective housing module 2.1, 2.2 (not shown).

While the exchange of both housing modules 2.1, 2.2 is shown in stylised form in embodiment example FIG. 1, the second housing module 2.2 is provided in the view from above in embodiment example FIG. 2, which is placed on the housing base body 2 in a modular design.

According to embodiment example FIG. 1, the earcup 1 also has a main shell module 3, which is connected to the housing base body 2 opposite the housing modules 2.1, 2.2. The main shell module 3 is primarily used to accommodate sound-absorbing means for the purpose of hearing protection. According to embodiment example FIG. 1, the main shell module 3 has an overall height S0.

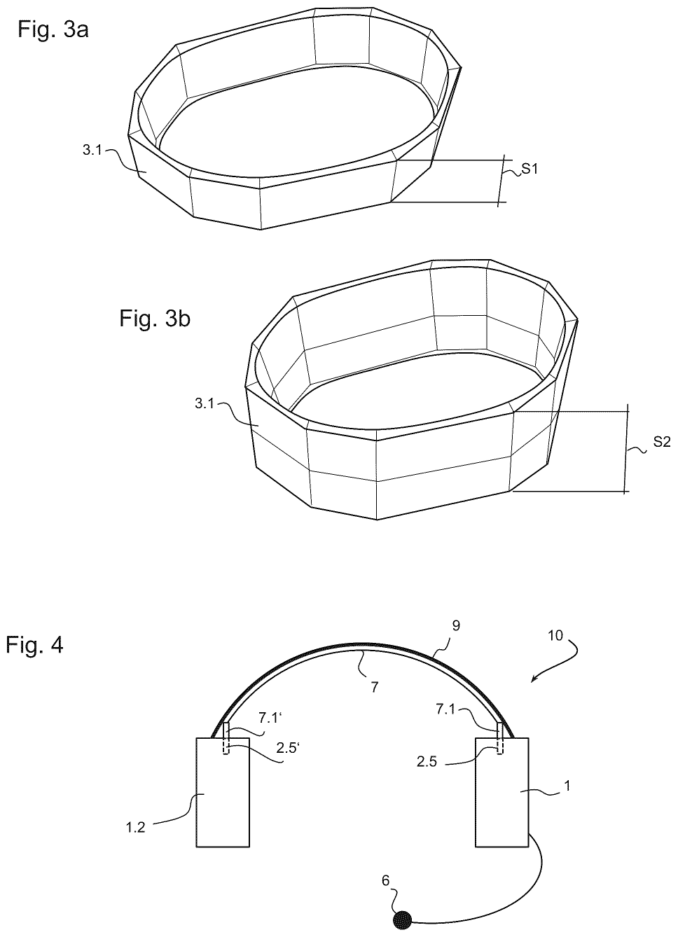

As an alternative or supplement to the main shell module 3, a first shell replacement module 3.1 is provided according to FIG. 3a with an overall height S1 that is approximately 30% greater than the overall height S0. The first shell replacement module 3.1 can be attached or fastened to the housing base body 2 in a modular design as an alternative to the main shell module 3. It is also intended that the first shell replacement module 3.1 is placed on or attached to the main shell module 3 in a modular design in addition to the main shell module 3. In both cases, the existing height S0 of the main shell module 3 is increased to a value S1 greater than 0 or to a value S0+S1.

A second shell replacement module 3.2 with a modular design is also provided. The second shell replacement module 3.2 has an overall height S2 that is approximately twice as large as the overall height S1. The second shell replacement module 3.2 can also be mounted on the main shell module 3 or on the first shell replacement module 3.1 in a modular design. Alternatively, the second shell replacement module 3.2 can be used in exchange for the main shell module 3 or in exchange for the first shell replacement module 3.1. In this way, a variety of heights can be achieved in a modular design for the purpose of adapting the desired sound attenuation.

Design example 4 shows the basic representation of headphones 10 with the earcup 1 and a second earcup 1.2. Both earcups 1, 1.2 are connected to each other via a headband 9 or are each attached to the end of the headband 9. Both earcups 1, 1.2 have the symmetry plane E shown in FIG. 1 and can therefore be used or attached in their position on the right and left of the headband 9. Both earcups 1, 1.2 are connected to each other via a connecting cable 7. The connecting cable 7 is electrically connected via a corresponding plug 7.1, 7.1′ to a corresponding plug socket 2.5, 2.5′ on the respective earcup 1, 1.2. The contact 2.6 formed in this way can be easily released manually to replace the respective earcup 1, 1.2. The earcup 1 provided on the right-hand side according to FIG. 4 also has a speaker microphone 6, which is attached or mounted on the housing base body 2 via the holder 5b shown in FIG. 1.

By forming a manually detachable contact 2.6 between the respective earcup 1, 1.2 and the connecting cable 7, it is possible to easily replace the respective components, i.e. the respective earcup 1, 1.2 or the connecting cable 7.

### LIST OF REFERENCE SYMBOLS

1 earcup
1.1 housing
1.2 second earcup
2 housing base body
2.1 first housing module
2.2 second housing module
2.3 microphone, microphone for ambient noise
2.4 microphone, microphone for ambient noise
2.5 plug socket, contact
2.5′ plug socket, contact
2.6 contact, plug-in contact
2.6′ contact, plug-in contact
3 main shell module
3.1 first shell replacement module
3.2 second shell replacement module
4a bearing mount
4b bearing mount
5a mount
5b mount
6 microphone, speaker microphone
7 connecting cable
7.1 plug
7.1′ plug
9 headband
10 headphones
E dymmetry plane
S0 overall height
S1 overall height
S2 overall height

What is claimed is:

1. An earcup for headphones, comprising:
a housing for accommodating electroacoustic components and sound attenuation material,
wherein the housing has a housing base body for accommodating electroacoustic components and a main shell module with an overall height S0 for accommodating a sound-absorbing material, the main shell module being mechanically connected to the housing base body, wherein
a) wherein for providing different expansion stages for acoustic damping, the housing has the main shell module, which is
a1) modularly exchangeable and at least one first shell replacement module with an overall height S1 is provided, that contains sound attenuation material and which can be mechanically connected to the housing base body as an alternative to the main shell module, wherein the overall heights S0 and S1 are different and/or;
a2) can be added in a modular manner and at least one first shell replacement module with an overall height S1 is provided, that contains sound attenuation material wherein the first shell replacement module can be mechanically connected to the main shell module, wherein the overall heights S0 and S1 are different, and/or
b) the housing base body has a symmetry plane E and is designed to be mirror-symmetrical with respect to the symmetry plane E, whereby the housing base body has at least one pair of mounts for a speaker microphone to be arranged outside the housing base body, the mounts being designed symmetrically with respect to the symmetry plane E.

2. The earcup according to claim 1, wherein the housing modules and/or the main shell module and/or the shell replacement modules have a symmetry plane E and are designed to be mirror-symmetrical with respect to the symmetry plane E.

3. The earcup according to claim 1, wherein the housing base body has at least one pair of bearing mounts for a headband, the bearing mounts being designed symmetrically with respect to the symmetry plane E.

4. The earcup according to claim 1, wherein the housing modules have electro-acoustic components and the housing base body has electro-acoustic components, wherein the electro-acoustic components can be brought into active contact with the electro-acoustic components.

5. The earcup according to claim 4, wherein a plug-in contact is provided, via which the active contact can be established.

6. The earcup according to claim 1, wherein the first housing module has at least one microphone as an electroacoustic component.

7. The earcup according to claim 1, that wherein at least one second shell module is provided which a) can be mechanically connected to the housing base body as an alternative to the main shell module or as an alternative to the first shell module, or b) can be mechanically connected to the first shell module.

8. The earcup according to claim 1, wherein the main shell module has an overall height S0 and the first shell replacement module has an overall height S1, wherein the overall heights S0 and S1 are different.

9. The earcup according to claim 7, wherein a second shell replacement module with an overall height S2 is provided, wherein the overall heights S0, S1 and S2 are different.

10. The earcup according to claim 8, wherein for the overall heights S0, S1 and/or S2 it applies that S1=f1*S0 and/or S2=f2*S0, with X1<f1<Y1 and X2<f2<Y2.

11. The earcup according to claim 1, wherein the housing base body has a plug socket for a plug of an electrical connecting cable to a second earcup.

12. Headphones comprising an earcup according to claim 1 and a second earcup, wherein the housing base body and/or the housing modules and/or the main shell module and/or the shell replacement modules of the two earcups have the same shape.

13. The earcup according to claim 2, wherein the housing base body has at least one pair of bearing mounts for a headband, the bearing mounts being designed symmetrically with respect to the symmetry plane E, wherein the housing modules have electro-acoustic components and the housing base body has electro-acoustic components, and wherein the electro-acoustic components can be brought into active contact with the electro-acoustic components.

14. The earcup according to claim 13, wherein a plug-in contact is provided, via which the active contact can be established, and wherein the first housing module has at least one microphone as an electroacoustic component.

15. The earcup according to claim 14, wherein at least one second shell module is provided which a) can be mechanically connected to the housing base body as an alternative to the main shell module or as an alternative to the first shell module, or b) can be mechanically connected to the first shell module, and wherein the main shell module has an overall height S0 and the first shell replacement module has an overall height S1, wherein the overall heights S0 and S1 are different.

16. The earcup according to claim 15, wherein a second shell replacement module with an overall height S2 is provided, wherein the overall heights S0, S1 and S2 are different, and wherein for the overall heights S0, S1 and/or S2 it applies that S1=f1*S0 and/or S2=f2*S0, with X1<f1<Y1 and X2<f2<Y2.

17. The earcup according to claim 16, wherein the housing base body has a plug socket for a plug of an electrical connecting cable to a second earcup.

18. Headphones comprising an earcup according to claim 17 and a second earcup, wherein the housing base body and/or the housing modules and/or the main shell module and/or the shell replacement modules of the two earcups have the same shape.
